(19) United States
(12) Patent Application Publication
(10) Pub. No.: US 2024/0269409 A1
(43) Pub. Date: Aug. 15, 2024

(54) SYSTEMS AND METHODS TO DETERMINE THE CONFIGURATION OF RESPIRATORY THERAPY SYSTEMS

(71) Applicant: ResMed Sensor Technologies Limited, Dublin (IE)

(72) Inventors: Redmond Shouldice, Dublin (IE); Graeme Alexander Lyon, Dublin (IE)

(21) Appl. No.: 18/435,366

(22) Filed: Feb. 7, 2024

Related U.S. Application Data
(60) Provisional application No. 63/484,388, filed on Feb. 10, 2023.

(51) Int. Cl. A61M 16/00; A61M 16/06; A61M 16/08

(52) U.S. Cl. CPC A61M 16/026; A61M 16/0069; A61M 16/0605; A61M 16/0875; A61M 2205/3334; A61M 2205/702

(57) ABSTRACT

A method includes generating a first airflow that travels from the respiratory therapy device to a user interface by operating a motor of the respiratory therapy device. The method also includes receiving first airflow parameter data associated with the first airflow. The method also includes determining a first user interface prediction, based on the first airflow parameter data. The method also includes generating a second airflow from the respiratory therapy device to the user interface by operating the motor of the respiratory therapy device. The method also includes receiving second airflow parameter data associated with the second airflow. The method also includes determining, based on the second airflow parameter data, a second user interface prediction. The method also includes characterizing the user interface based at least in part on the first user interface prediction and the second user interface prediction.

### CROSS-REFERENCE TO RELATED APPLICATIONS

This application claims the benefit of, and priority to, U.S. Provisional Patent Application No. 63/484,388, filed Feb. 10, 2023, which is hereby incorporated by reference herein in its entirety.

### TECHNICAL FIELD

The present disclosure relates generally to systems and methods for configuration of respiratory therapy systems, and more particularly, to systems and methods for automatically characterizing a user interface of a respiratory therapy system, which characterization may be used to determine the configuration of the respiratory therapy system.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often treated using a respiratory therapy system (e.g., a continuous positive airway pressure (CPAP) system), which delivers pressurized air to aid in preventing the individual's airway from narrowing or collapsing during sleep. However, some users find such systems to be uncomfortable, difficult to use, expensive, aesthetically unappealing and/or fail to perceive the benefits associated with using the system.

In addition to setting up a respiratory therapy system, difficulties in selecting the most suitable mask type or size, difficulties in achieving good mask seal, etc., can all contribute to a user's reluctance to begin or persist with respiratory therapy. It is therefore advantageous to know the user interface (mask) connected, such as via a conduit, to a respiratory therapy device. This information will help facilitate set up of the respiratory therapy system and facilitate improved control of therapy delivered to the user. For instance, it is advantageous to know the user interface for the selection of treatment parameters, such as the airflow pressure at the mask. Accordingly, knowledge of what user interface is being used by the user of the respiratory therapy system can enhance therapy efficacy as well as the user's experience of therapy. The present disclosure is directed to solving these and other problems.

### SUMMARY

According to some implementations of the present disclosure, a method for characterizing a user interface includes generating a first airflow while the user interface is not donned by a user. The user interface is coupled, via a conduit, to a respiratory therapy device in a respiratory therapy system. The first airflow travels from the respiratory therapy device to the user interface by operating a motor of the respiratory therapy device. The method also includes receiving first airflow parameter data associated with the first airflow. The method also includes determining a first user interface prediction. The determination is based at least in part on the first airflow parameter data. The method also includes generating, while the user interface is donned by the user, a second airflow from the respiratory therapy device to the user interface by operating the motor of the respiratory therapy device. The method also includes receiving second airflow parameter data associated with the second airflow. The method also includes determining, based at least in part on the second airflow parameter data, a second user interface prediction. The method also includes characterizing the user interface based at least in part on the first user interface prediction and the second user interface prediction.

According to some implementations of the present disclosure, a respiratory therapy system includes a respiratory therapy device, a conduit, and a user interface. The user interface is coupled to the respiratory therapy device via the conduit. The system also includes a memory device, and a control system. The memory stores machine-readable instructions. The control system includes one or more processors configured to execute the machine-readable instructions to cause, while the user interface is not donned by a user, a first airflow to be generated from the respiratory therapy device to the user interface by operating a motor of the respiratory therapy device. The control system is further configured to receive first airflow parameter data associated with the first airflow. The control system is further configured to determine a first user interface prediction based at least in part on the first airflow parameter data. The control system is further configured to cause, while the user interface is donned by the user, a second airflow to be generated from the respiratory therapy device to the user interface by operating the motor of the respiratory therapy device. The control system is further configured to receive second airflow parameter data associated with the second airflow. The control system is further configured to determine a second user interface prediction based at least in part on the second airflow parameter data. The control system is further configured to characterize the user interface based at least in part on the first user interface prediction and the second user interface prediction.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2A is a perspective view of at least a portion of the system of FIG. 1, a user, and a bed partner, according to some implementations of the present disclosure;

FIG. 2B is a perspective view of at least a portion of the system of FIG. 1, a user donning a user interface, and a bed partner, according to some implementations of the present disclosure;

FIG. 3A is a perspective view of a user interface, according to some implementations of the present disclosure;

FIG. 3B is an exploded view of the user interface of FIG. 3A, according to some implementations of the present disclosure;

FIG. 4 illustrates an exemplary timeline for a sleep session, according to some implementations of the present disclosure;

FIG. 5 illustrates an exemplary hypnogram associated with the sleep session of FIG. 4, according to some implementations of the present disclosure;

FIG. 6A is a process flow diagram for a method for characterizing a user interface of a respiratory therapy system according to some implementations of the present disclosure;

FIG. 6B is a process flow diagram for a method for determining unintentional leak from a user interface according to some implementations of the present disclosure;

FIG. 6C is a process flow diagram for a method for operating a motor of a respiratory therapy device at a variety of different speeds according to some implementations of the present disclosure;

FIG. 7A is a representational view of a pressure versus airflow graph used in the process of calculating a characteristic curve according to some implementations of the present disclosure;

FIG. 7B is a representational view of a pressure versus airflow graph used in the process of calculating a characteristic curve according to some implementations of the present disclosure;

FIG. 7C is a representational view of a pressure versus airflow graph used in the process of calculating a characteristic curve according to some implementations of the present disclosure;

FIG. 7D is a representational view of a pressure versus airflow graph used in the process of calculating a characteristic curve according to some implementations of the present disclosure; and

FIG. 8 is a representational view of a pressure versus airflow graph having pre-defined characteristic curves according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

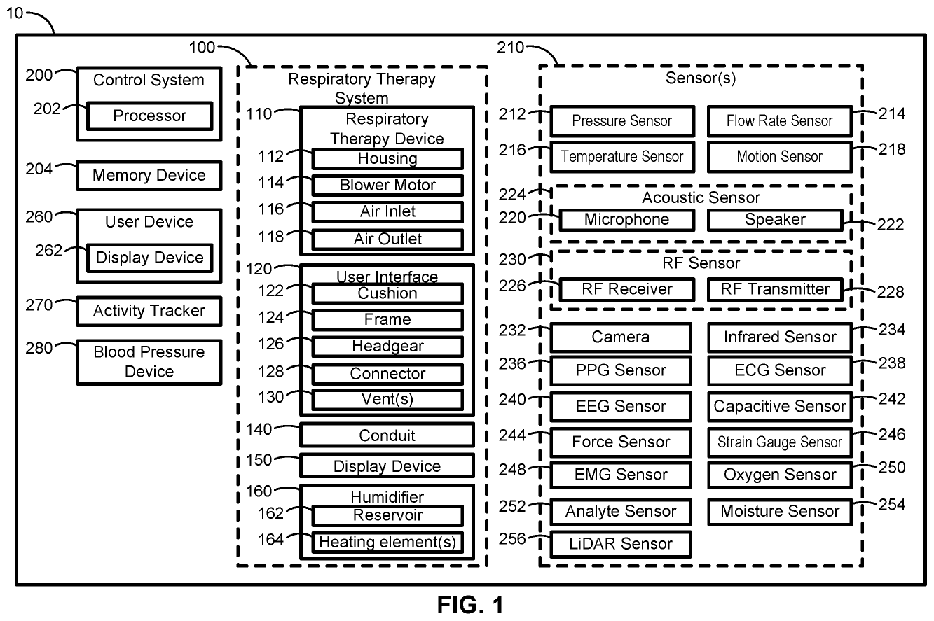

Referring to FIG. 1, a system 10, according to some implementations of the present disclosure, is illustrated. The system 10 includes a respiratory therapy system 100, a control system 200, one or more sensors 210, a user device 260, and an activity tracker 270.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

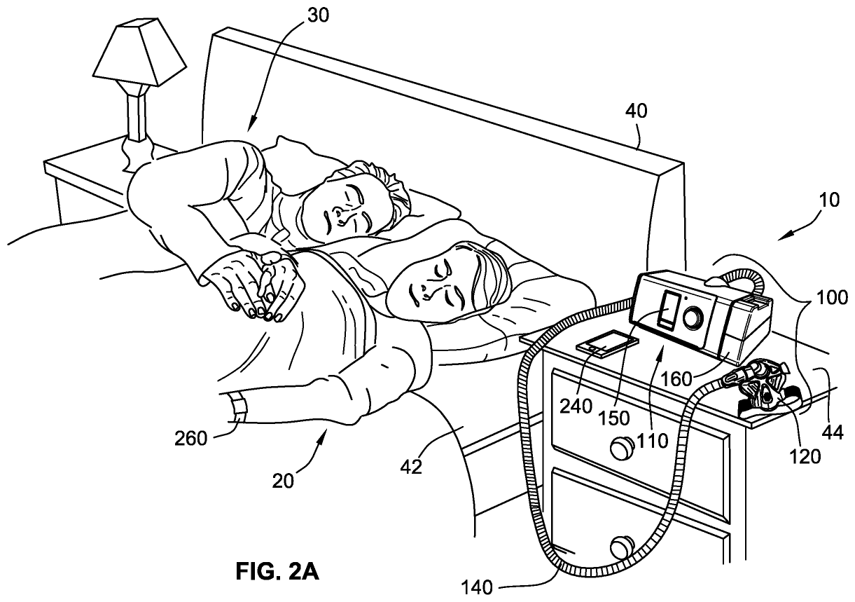

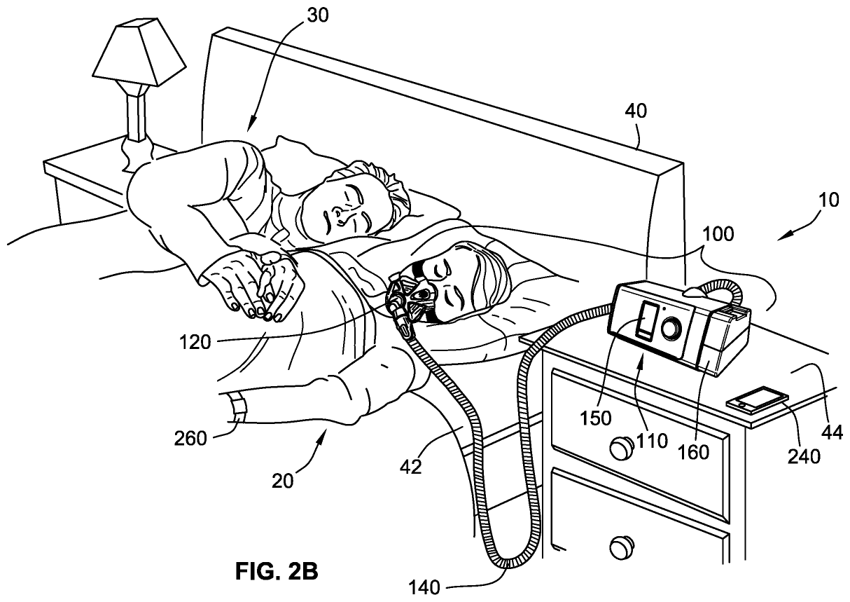

As shown in FIGS. 2A-2B, the respiratory therapy system 100 can be used to treat user 20. In this example, the user 20 of the respiratory therapy system 100 and a bed partner 30 are located in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIGS. 2A-2B, or more generally, on any surface or structure that is generally adjacent to the bed 40 and/or the user 20. The user interface 120 may also be positioned on the nightstand 44 when not donned by the user 20, e.g., as seen in FIG. 2A. However, it should be noted that the user interface 120 and/or any portion of the cable 140 may be placed in any desired location while not donned by the user, e.g., in a carrying case for the respiratory therapy device 110, in a drawer of the nightstand 44, on the floor next to the bed 40, etc.

The respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about −20 L/min and about 150 L/min, while maintaining a positive pressure (relative to the ambient pressure). The respiratory therapy device 110 also includes a housing 112, a blower motor 114, an air inlet 116, and an air outlet 118 (FIG. 1).

Referring still to FIG. 1, the user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The vent 130 can be used for permitting the escape of carbon dioxide and other gases exhaled by the user 20. The user interface 120 generally can include any suitable number of vents (e.g., one, two, five, ten, etc.).

As shown in FIG. 2B, in some implementations, the user interface 120 is a facial mask (e.g., a full face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

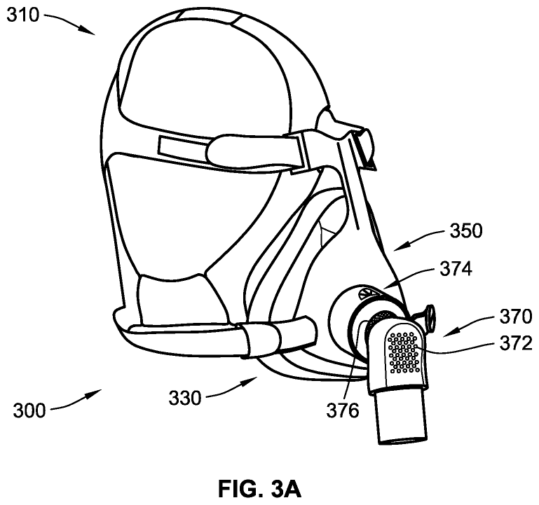

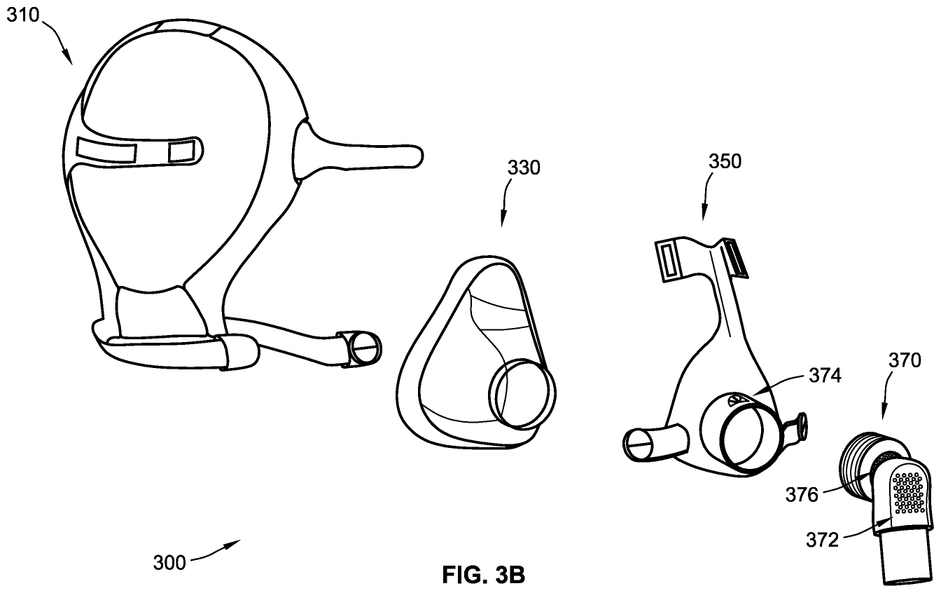

Referring to FIGS. 3A and 3B, a user interface 300 that is the same as, or similar to, the user interface 120 (FIG. 1) according to some implementations of the present disclosure is illustrated. The user interface 300 generally includes a cushion 330 and a frame 350 that define a volume of space around the mouth and/or nose of the user. When in use, the volume of space receives pressurized air for passage into the user's airways. In some implementations, the cushion 330 and frame 350 of the user interface 300 form a unitary component of the user interface. The user interface 300 can also include a headgear 310, which generally includes a strap assembly and optionally a connector 370. The headgear 310 is configured to be positioned generally about at least a portion of a user's head when the user wears the user interface 300. The headgear 310 can be coupled to the frame 350 and positioned on the user's head such that the user's head is positioned between the headgear 310 and the frame 350. The cushion 330 is positioned between the user's face and the frame 350 to form a seal on the user's face. The optional connector 370 is configured to couple to the frame 350 and/or cushion 330 at one end and to a conduit of a respiratory therapy device (not shown). The pressurized air can flow directly from the conduit of the respiratory therapy system into the volume of space defined by the cushion 330 (or cushion 330 and frame 350) of the user interface 300 through the connector 370). From the user interface 300, the pressurized air reaches the user's airway through the user's mouth, nose, or both. Alternatively, where the user interface 300 does not include the connector 370, the conduit of the respiratory therapy system can connect directly to the cushion 330 and/or the frame 350.

In some implementations, the connector 370 may include one or more vents 372 (e.g., a plurality of vents) located on the main body of the connector 370 itself and/or one or a plurality of vents 376 (“diffuser vents”) in proximity to the frame 350, for permitting the escape of carbon dioxide (CO₂) and other gases exhaled by the user. In some implementations, one or a plurality of vents, such as vents 372 and/or 376 may be located in the user interface 300, such as in frame 350, and/or in the conduit 140. In some implementations, the frame 350 includes at least one anti-asphyxia valve (AAV) 374, which allows CO₂ and other gases exhaled by the user to escape in the event that the vents (e.g., the vents 372 or 376) fail when the respiratory therapy device is in use. In general, AAVs (e.g., the AAV 374) are present for full face masks (e.g., as a safety feature); however, the diffuser vents and vents located on the mask or connector (usually an array of orifices in the mask material itself or a mesh made of some sort of fabric, in many cases replaceable) are not necessarily both present (e.g., some masks might have only the diffuser vents such as the plurality of vents 376, other masks might have only the plurality of vents 372 on the connector itself).

As noted above, the specific details shown in FIGS. 2A-3B are in no way intended to be limiting. Rather, other implementations may include user interfaces that are the same as, similar to, or different from the user interfaces 120 depicted in FIGS. 2A-3B. For instance, some user interfaces are indirect user interfaces, while other user interfaces are direct user interfaces. An indirectly connected user interface may deliver pressurized air from the conduit 140 of the respiratory therapy system to the cushion and/or frame through a user interface conduit(s), rather than directly from the conduit 140 of the respiratory therapy system.

Referring back to FIG. 1, the conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

In some implementations, the conduit 140 includes a first end that is coupled to the air outlet 118 of the respiratory therapy device 110. The first end can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a myAir™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. For example, air may flow from an air inlet through a blower motor, and then through a humidifier before exiting the respiratory therapy device 110 via air outlet 118.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct).

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

Referring to back to FIG. 1, the one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a LiDAR sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the infrared sensor 234, the photoplethysmogram (PPG) sensor 236, the electrocardiogram (ECG) sensor 238, the electroencephalography (EEG) sensor 240, the capacitive sensor 242, the force sensor 244, the strain gauge sensor 246, the electromyography (EMG) sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 (FIGS. 2A-2B) during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20 (FIGS. 2A-2B), a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 alternatively or additionally generates one or more signals representing bodily movement of the user, from which may be obtained a signal representing a sleep state of the user; for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones.

The speaker 222 outputs sound waves that are audible to a user of the system 10 (e.g., the user 20 of FIGS. 2A-2B). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260.

The microphone 220 and the speaker 222 can be used as separate devices. In some implementations, the microphone 220 and the speaker 222 can be combined into an acoustic sensor 224 (e.g., a SONAR sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30 (FIGS. 2A-2B). Based at least in part on the data from the microphone 220 and/or the speaker 222, the control system 200 can determine a location of the user 20 (FIGS. 2A-2B) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g., a RADAR sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user (FIGS. 2A-2B), to determine air flow of the mouth and/or nose of the user, to determine a time when the user enters the bed (FIGS. 2A-2B), and to determine a time when the user exits the bed. In some implementations, the camera 232 includes a wide angle lens or a fish eye lens.

The infrared (IR) sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 (FIGS. 2A-2B) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120 and/or the associated headgear (e.g., straps, etc.).

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 174 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 174 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The Light Detection and Ranging (LiDAR) sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 100, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 132 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, a microphone, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 (FIG. 1) includes a display device 262. The user device 260 can be, for example, a mobile device such as a smart phone, a tablet, a gaming console, a smart watch, a laptop, or the like. Alternatively, the user device 260 can be an external sensing system, a television (e.g., a smart television) or another smart home device (e.g., a smart speaker(s) such as Google Home™, Google Nest™, Amazon Echo™, Amazon Alexa™-enabled devices, etc.). In some implementations, the user device is a wearable device (e.g., a smart watch, a ring, a wristband, etc.). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 100 also includes an activity tracker 270. The activity tracker 270 is generally used to aid in generating physiological data associated with the user. The activity tracker 270 can include one or more of the sensors 210 described herein, such as, for example, the motion sensor 138 (e.g., one or more accelerometers and/or gyroscopes), the PPG sensor 154, and/or the ECG sensor 156. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIGS. 2A-2B, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 100 also includes a blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIGS. 2A-2B, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp, chirp, triangle, sinewave, etc.), other schemes such as PSK, FSK etc., pulsed continuous wave, and/or spread in ultra wideband ranges (which may include spreading, PRN codes or impulse systems).

While the control system 200 and the memory device 204 are described and shown in FIG. 1 as being a separate and distinct component of the system 100, in some implementations, the control system 200 and/or the memory device 204 are integrated in the user device 260 and/or the respiratory therapy device 110. Alternatively, in some implementations, the control system 200 or a portion thereof (e.g., the processor 202) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user 20 has laid or sat down in the bed 40 (or another area or object on which they intend to sleep), and has turned on the respiratory therapy device 110 and donned the user interface 120. The sleep session can thus include time periods (i) when the user 20 is using the respiratory therapy system 100, but before the user 20 attempts to fall asleep (for example when the user 20 lays in the bed 40 reading a book); (ii) when the user 20 begins trying to fall asleep but is still awake; (iii) when the user 20 is in a light sleep (also referred to as stage 1 and stage 2 of non-rapid eye movement (NREM) sleep); (iv) when the user 20 is in a deep sleep (also referred to as slow-wave sleep, SWS, or stage 3 of NREM sleep); (v) when the user 20 is in rapid eye movement (REM) sleep; (vi) when the user 20 is periodically awake between light sleep, deep sleep, or REM sleep; or (vii) when the user 20 wakes up and does not fall back asleep.

The sleep session is generally defined as ending once the user 20 removes the user interface 120, turns off the respiratory therapy device 110, and/or gets out of bed 40. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user 20, ending when the respiratory therapy device 110 stops supplying the pressurized air to the airway of the user 20, and including some or all of the time points in between, when the user 20 is asleep or awake.

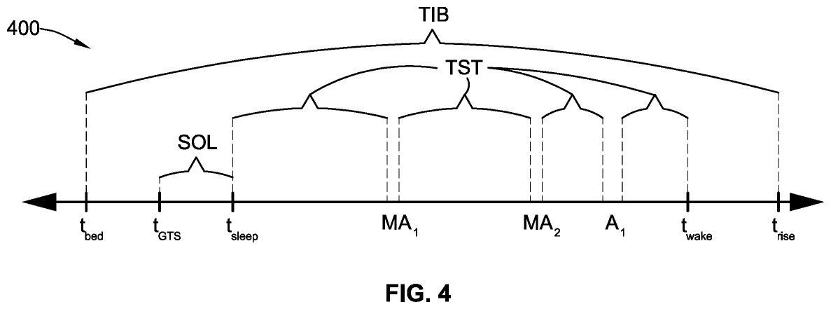

Referring to the timeline 400 in FIG. 4 the enter bed time t_{bed} is associated with the time that the user initially enters the bed (e.g., bed 40 in FIGS. 2A-2B) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 400 of FIG. 4, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

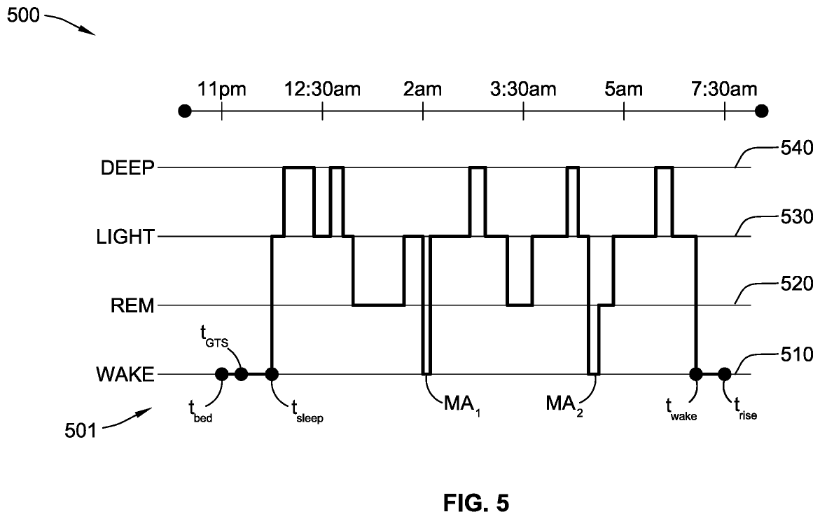

Referring to FIG. 5, an exemplary hypnogram 500 corresponding to the timeline 400 (FIG. 4), according to some implementations, is illustrated. As shown, the hypnogram 500 includes a sleep-wake signal 501, a wakefulness stage axis 510, a REM stage axis 520, a light sleep stage axis 530, and a deep sleep stage axis 540. The intersection between the sleep-wake signal 501 and one of the axes 510-540 is indicative of the sleep stage at any given time during the sleep session.

The sleep-wake signal 501 can be generated based on physiological data associated with the user (e.g., generated by one or more of the sensors 210 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 500 is shown in FIG. 5 as including the light sleep stage axis 530 and the deep sleep stage axis 540, in some implementations, the hypnogram 500 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 204.

The hypnogram 500 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 218, the microphone 220, the camera 232, or any combination thereof. The go-to-sleep time can be determined based on, for example, data from the motion sensor 218 (e.g., data indicative of no movement by the user), data from the camera 232 (e.g., data indicative of no movement by the user and/or that the user has turned off the lights) data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

As noted above, a user interface typically engages a portion of the user's face and delivers pressurized air from a respiratory therapy device to the user's airway to aid in preventing the airway from narrowing and/or collapsing during sleep. The pressure, humidity, flow rate, etc., of the air (also referred to herein as the “properties of the air”) provided to the user thereby has a significant impact on the therapy received by the user, as well as the quality of sleep experienced by the user during a sleep session while donning the user interface.

While particular properties of the air provided to a user donning the user interface may be known for a particular implementation, the properties of the air that is actually delivered to the user interface may differ from properties of the air when it was generated by the respiratory therapy device. In other words, factors associated with how the air generated by the respiratory therapy device is physically delivered to the user's mouth and/or nose has an impact on the properties of the air that is actually provided to the user donning the user interface. These factors may include, but are in no way limited to, the configuration of the conduit connecting the user interface to the respiratory therapy device (e.g., the length, diameter, etc.), the configuration of the user interface itself (e.g., pressurized air is delivered to the user's mouth, the user's nose, or both the user's mouth and nose via different mask types, e.g., nasal, pillows, full-face, etc.), the manner in which air is supplied to the user (e.g., using an indirect user interface, a direct user interface, etc.), etc.

Previous therapy architectures have relied on users correctly programming their specific mask into a therapy device in order to receive desired properties of air during a sleep session. However, some differences between mask types are not easily recognizable and make it difficult for users to correctly identify their mask. This, in combination with other contributing issues (e.g., such as a user forgetting to update a mask setting after attaching a new mask of a different type), has caused user confusion and frustration, ultimately reducing the quality of therapy, comfort and/or sleep experienced by users.

In sharp contrast to these conventional shortcomings, various ones of the implementations included herein are able to automatically detect specific components that are being used in a respiratory therapy system. In other words, various ones of the implementations included herein are able to accurately identify factors associated with how the air generated by the respiratory therapy device is physically delivered to the user. For instance, implementations herein are able to seamlessly detect a mask type, cushion size, conduit type, etc., that is currently in use. The respiratory therapy system is thereby able to automatically detect the components in use, and adjust operation of the various components therein (e.g., motor, humidifier, etc.) to provide air having a specific pressure, humidity, flow rate, etc., to the user that ultimately improves the quality of therapy provided to the user and/or the sleep experienced by the user during therapy. This also desirably simplifies the interaction and improves the overall user experience.

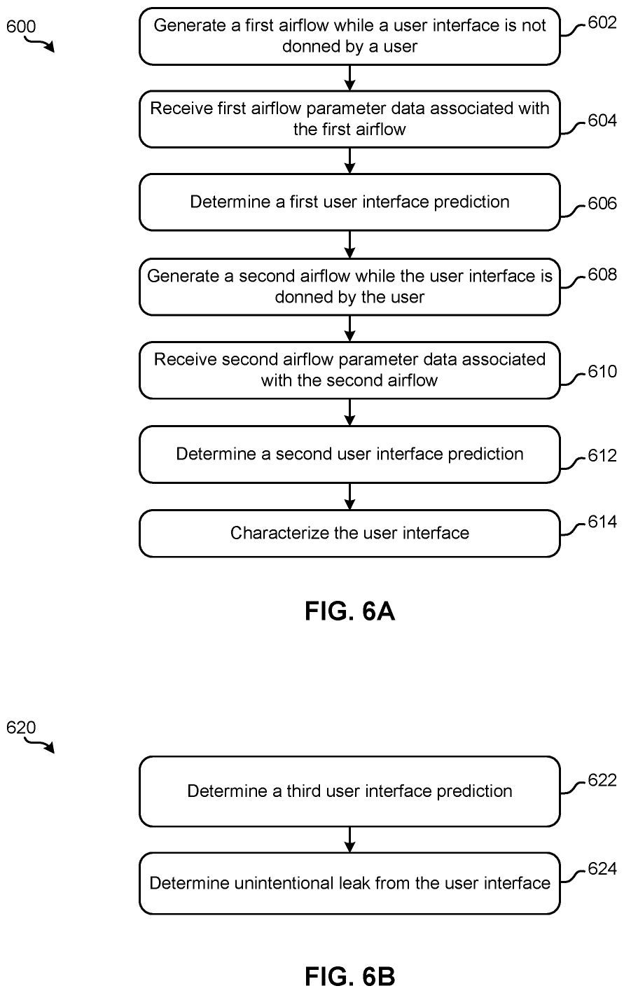

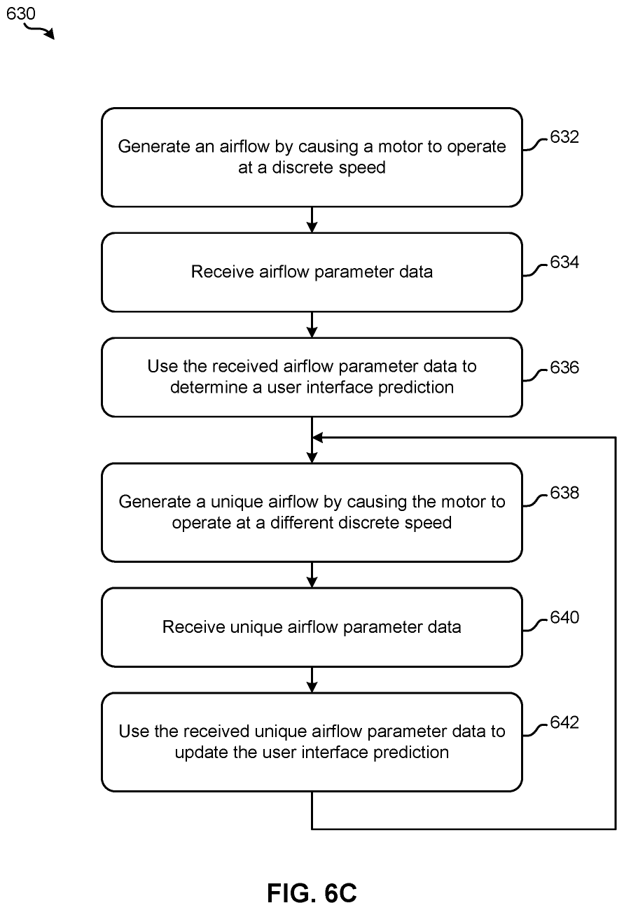

Referring to FIG. 6A, a method 600 for characterizing a user interface of a respiratory therapy system according to some implementations of the present disclosure is illustrated. By causing the respiratory therapy device to generate specific airflows via the conduit to the user interface, method 600 is able to automatically detect the specific components being used in a respiratory therapy system. While the configuration of the respiratory therapy system may differ depending on the implementation, method 600 has been presented below in the context of a system having a user interface that is coupled to a respiratory therapy device via a conduit (e.g., see FIGS. 2A-2B). It follows that one or more steps of the method 600 can be implemented using any element or aspect of the system 100 (FIGS. 1-2B) described herein.

More or less operations than those specifically described in FIG. 6A may be included in method 600, as would be understood by one of skill in the art upon reading the present descriptions. Each of the steps of the method 600 may be performed by any suitable component of the operating environment. For example, in various implementations, the method 600 may be partially or entirely performed by a controller, a processor, a computer, etc., or some other device having one or more processors therein. Thus, in some implementations, method 600 may be a computer-implemented method. Moreover, the terms computer, processor and controller may be used interchangeably with regards to any of the implementations herein, such components being considered equivalents in the many various permutations of the present invention.

Moreover, for those implementations having a processor, the processor, e.g., processing circuit(s), chip(s), and/or module(s) implemented in hardware and/or software, and preferably having at least one hardware component may be utilized in any device to perform one or more steps of the method 600. Illustrative processors include, but are not limited to, a central processing unit (CPU), an application specific integrated circuit (ASIC), a field programmable gate array (FPGA), etc., combinations thereof, or any other suitable computing device known in the art.

As shown in FIG. 6A, operation 602 of method 600 includes generating a first airflow while the user interface is not donned by a user. Thus, the first airflow may serve as a pseudo open-circuit impedance. With respect to the present description, the first airflow is generated at the respiratory therapy device and moves through the conduit to the user interface. It follows that in some implementations, the first airflow may actually be generated by sending one or more instructions (e.g., commands, requests, etc.) to a motor in the respiratory therapy device.

By generating the first airflow while the user interface is not donned by the user, the respiratory therapy device may be operated without concern for a comfort level of the user. Components such as a motor in the respiratory therapy device may thereby be set to run at one or more speeds that produce noise, heat, vibrations, air properties (such as flow rate and pressure), etc., that would otherwise be undesirable to a user donning the user interface (e.g., see FIG. 6C below). For example, abrupt adjustments to settings of the user interface and/or respiratory therapy systems as a whole may be less desirable for a user donning the user interface. It follows that the term “undesirable” as used herein may refer to an abrupt (e.g., sharp) transition in any operating setting thereof. For example, motor speeds over a certain value (e.g., great than 20,000, greater than between 25,000 RPM and 30,000 RPM, greater than 40,000 RPM, etc.) may result in uncomfortable airflow conditions for a user. According to another example, an abrupt increase in air pressure experienced by a user may be highly uncomfortable. Rather, it is more desirable in some implementations to adjust operating settings according to a predetermined rate or range. This value or range may even be dynamically updated as user preferences change over time, software updates are implemented, etc. This allows for the system to collect information associated with a variety of different operating settings that can be used to characterize the various components included in the system, e.g., as will be described in further detail below.

It follows that the first airflow may be generated so as to have one or more desired properties. For example, the first airflow may have a specific pressure and/or flow rate as a result of operating a motor of the respiratory therapy device at one or more particular speeds. The one or more speeds at which the motor operates while the user interface is not donned by the user may be higher or lower than a nominal (e.g., average) speed of the motor while the user interface is donned by the user. For instance, in some implementations the motor may operate at one or more speeds that are less than an average operating speed while the user interface is donned by a user.

According to an example, that is in no way intended to limit the invention, the motor may operate at speeds that are at least 1%, 5%, 6%, 7%, 10%, 15%, 20%, 30%, 40%, 50%, 75%, etc., below an average operating speed of the motor while the user interface is donned by a user. This may be useful to achieve pressures and/or flow rates suitable to detect the presence of an AAV (and thereby characterize the user interface as a full face mask), such as by causing the AAV to close (and optionally open and close repeatedly for a duration of time), which may be detected acoustically, for example, as described in WO 2022/219481A1, which is hereby incorporated by reference in its entirety. In another example, which is in no way intended to limit the invention, the motor may operate at speeds that are less than 1%, 5%, 6%, 7%, 10%, 15%, 20%, 30%, etc., above a minimum operating speed of the motor. However, in other implementations the motor may operate at one or more speeds that are greater than an average operating speed while the user interface is donned by a user. According to an example, which again is in no way intended to limit the invention, the motor may operate at speeds that are at least 1%, 5%, 6%, 7%, 10%, 15%, 20%, 30%, 40%, 50%, 75%, etc., above an average operating speed of the motor while the user interface is donned by a user. This may be useful to achieve pressures and/or flow rates suitable to characterize a vent type, such as by causing air to pass through the vents, which may generate an acoustic signature unique to the vent and/or mask type, as described in the aforementioned WO 2022/219481A1.

In another example, an operating speed of the motor itself may be used to control performance. For instance, the motor may operate at speeds of between about 3,000 revolutions per minute (RPM) and about 12,000 RPM (which correspond to speeds that are typically below those associated with providing adequate pressure and flow for respiratory therapy, but which result in little or no perceptible noise from the motor), but could be higher or lower depending on the implementation. As noted above, this may be at least somewhat slower than an initial speed of the motor. For example, the motor may have an initial (e.g., nominal, average, starting, etc.) speed of about 20,000 RPM, but could be higher or lower. In still other implementations, the motor may be configured to produce pseudo-random noise (PRN) by implementing a dither with the motor. This may desirably allow for acoustic and/or other types of data to be collected while also reducing the amount of ambient noise produced as a result. It follows that motors may be operated at any desired number of speeds, for any desired amount(s) of time, at any desired frequency, etc.

According to another example, the first airflow may have a desired humidity level as a result of operating a humidifier of the respiratory therapy device at one or more specific settings. In some implementations, the first airflow may even be generated for a specific (e.g., predetermined) amount of time. This amount of time may correspond to one or more testing protocols that may be performed to characterize a user interface of a respiratory therapy system. In other implementations, the amount of time the first airflow is generated corresponds to one or more of a predetermined time, a user attempting to don the user interface, a quality of sleep experienced by the user during a last sleep session, etc. Weight sensors may also be used to detect a relative weight of the humidifier and/or a water reservoir used by the humidifier. Accordingly, the user may be prompted to fill the water reservoir prior to a sleep session to avoid interrupted sleep.

It should also be noted that, with respect to the present description, a user interface is considered “donned by the user” in situations where the user interface is coupled to a face of the user as designed (e.g., intended), and the user interface is considered “not donned by the user” at least in situations where the user interface is not coupled to a face of the user as designed. For example, a user interface laying on a nightstand next to a bed is not donned by the user (e.g., see FIG. 2A), as well as a user interface that the user is merely holding in their hands. However, according to another example, a user interface having headgear coupled to a user's head and a cushion engaging the user's face such that the respiratory therapy device is able to provide air having specific properties to the user is considered donned by the user (e.g., see FIG. 2B).

In some implementations, the user device may be used to prompt the user on whether or not the user interface should be donned at a given point in time. For example, the user device 260 may display a pop-up window, via display device 262, that displays a prompt that updates to inform the user whether the user interface should be donned at the moment or a future window of time. In other implementations, one or more instructions, requests, prompts, etc., may be sent to an electronic device associated with the user, to the user interface or conduit (such as to illuminate an integrated light), and/or to a display of the respiratory therapy system. For example, instructions to don and/or remove the user interface may be sent to a user's mobile device, smart watch, etc. These instructions may correspond to different testing periods implemented by the system, thereby ensuring that information received from various sensors is interpreted in the appropriate context. However, in other implementations, the respiratory therapy system is able to automatically detect whether the user interface is currently donned by a user (such as by detecting respiration by the user through the user interface), and adjust operating conditions of the respiratory therapy device accordingly.

Referring still to FIG. 6A, operation 604 includes receiving first airflow parameter data associated with the first airflow generated in operation 602. The airflow parameter data may be received from one or more sensors associated with the respiratory therapy device. Moreover, this airflow parameter data may be used to determine various details associated with the present implementation of the respiratory therapy system. For instance, in some instances the airflow parameter data may be used to confirm that the user interface is not donned by the user at a given point in time. In other instances, the airflow parameter data may be used to determine one or more of the components that are implemented in the respiratory therapy system. According to an example, the airflow parameter data may be used to determine the type, model, and/or size, etc. of the user interface included in (e.g., coupled to) the respiratory therapy device.

The one or more sensors from which the first airflow parameter data is received may be coupled to various portions of the respiratory therapy device, positioned in a same room as the respiratory therapy device, configured to communicate with one or more components in the respiratory therapy device, etc. According to an example, which is in no way intended to limit the invention, acoustic-based airflow parameter data may be received from one or more transducers that are configured to convert sound waves into electrical signals. These transducers may be positioned on a surface (e.g., inner surface) of the user interface, along the conduit, in the respiratory therapy device in acoustic communication with the flow of air, adjacent to a motor of the respiratory therapy device, etc., such that sound waves associated with a user breathing into the user interface and/or airflow being generated may be identified in real-time.

Various types of airflow parameter data received from at least some sensors may be stored in memory over time and even analyzed to develop an accurate understanding of how the system performs during various types of use. For instance, one or more samples may be received from the sensors while intentionally operating the respiratory therapy device in certain settings. According to an example, a user may be prompted to keep the user interface off their face during a first testing period configured to operate the respiratory therapy device in a variety of different settings. Sensors are thereby able to collect various types of airflow parameter data (e.g., acoustic information, temperature information, pressure information, flow information, humidity information, etc.) associated with how the system operates while the user interface is not donned by the user. Accordingly, the first airflow data may include any one or more of acoustic data, flow data, pressure data, impedance data associated with the first airflow passing through the conduit, etc. Similarly, a user may be prompted to place the user interface on their face during a second testing period configured to operate the respiratory therapy device in a variety of different settings. The sensors are thereby also able to collect a variety of airflow parameter data associated with how the system operates while the user interface is donned by the user.

It follows that the airflow parameter data received in operation 604 preferably includes data for the first airflow(s) generated in operation 602. In other words, each unique operating condition of the respiratory therapy device is preferably maintained long enough for rich data samples associated with the given operating condition to be collected by the various sensors. According to an example, which is in no way intended to limit the invention, a motor in the respiratory therapy device may produce a specific number of revolutions per minute for a period of time sufficient for sensors in and/or around the respiratory therapy device to collect rich data associated with how the various other components in the respiratory therapy device operate at a specific air pressure, e.g., as would be appreciated by one skilled in the art after reading the present description. It should be noted that, with respect to the present description, “rich data” is intended to refer to any detailed (e.g., in-depth) data that may be used to make complex determinations. For example, data that may be used to predict consumer (e.g., user) behavior may be considered “rich data” in some instances. In other instances, data acquired in relation to performing qualitative research may be considered “rich data,” e.g., as would be appreciated by one skilled in the art after reading the present description.

Again, this airflow parameter data may be used to better understand the operating conditions of the system and automatically determine certain information, e.g., such as the type of user interface being used, whether the user interface is currently donned by the user, a type of conduit coupling the user interface to the respiratory therapy device, etc. According to an example, information received from one or more sensors that are coupled to various portions of the respiratory therapy device, positioned in a same room as the respiratory therapy device, configured to communicate with one or more components in the respiratory therapy device, etc. may be used to identify a specific type of user interface and/or conduit included in the respiratory therapy device. As noted above, while specific properties of the air provided to a user donning the user interface may be preferred for a particular implementation, the properties of the air that is actually delivered to the user interface may differ from properties of the air when it was generated by the respiratory therapy device. In other words, factors associated with how the air generated by the respiratory therapy device is physically delivered to the user's mouth and/or nose has an impact on the properties of the air that is actually provided to the user donning the user interface.

Previous therapy architectures have relied on users correctly programming their specific mask into a device in order to receive desired properties of air during a sleep session. However, some differences between mask types are not easily recognizable and make it difficult for users to correctly identify their mask. This, in combination with other contributing situations (e.g., such as a user forgetting to update a mask setting after attaching a new mask of a different type), have caused user confusion and frustration, ultimately reducing the quality of therapy, comfort and/or sleep experienced by users in conventional instances.

As noted above, various ones of the implementations included herein are able to overcome the conventional shortcomings, by automatically detecting specific components that are being used in a respiratory therapy system, and even how they are being used. In other words, various ones of the implementations included herein are able to accurately identify factors associated with how the air generated by the respiratory therapy device is physically delivered to the user. For instance, implementations herein are able to seamlessly detect a user interface type, cushion size, conduit type, whether a user interface is donned by a user, etc. The respiratory therapy system is thereby able to automatically detect the components in use, and adjust operation of the various components therein (e.g., motor, humidifier, etc.) to provide air to the user that ultimately improves the quality of therapy provided to the user and/or the sleep experienced by the user.

With continued reference to FIG. 6A, operation 606 includes determining a first user interface prediction. In other words, operation 606 includes using the airflow parameter data received in operation 604 and/or information associated with the first airflow(s) generated in operation 602 to make a prediction as to the type of user interface that is coupled to the respiratory therapy device via the conduit. This first user interface prediction may be made using any desired information, processes, inputs, etc.

According to some implementations, one or more characteristic curves may be used to make the first user interface prediction. In other words, operation 606 may involve determining a characteristic curve that best matches at least some of the airflow parameter data received. Because the first airflow is generated while the user interface is not donned by the user in preferred implementations as described above, the first characteristic curve also corresponds to the user interface not being donned by the user. It follows that the first characteristic curve may be determined prior to a beginning of a sleep session. This determination of the first characteristic curve includes calculating the first characteristic curve prior to the beginning of the sleep session.

Characteristic curves may be used to represent how the user interface and/or conduit functions with respect to two or more characteristics. According to an example, which is in no way intended to limit the invention, a characteristic curve as referenced herein may be a pressure versus flow curve. In another example, a characteristic curve as referenced herein may be an impedance versus flow curve. In another example, which again is in no way intended to limit the invention, a characteristic curve may include acoustic data relating to (use of) the respiratory therapy system. It follows that any of the characteristic curves included herein may present airflow parameter data in any desired manner. Even use of the term “curve” should in no way be limiting. In other words, a “characteristic curve” as used herein may include information that is presented in any desired way, e.g., such as graphs that do not include curves (e.g., bar charts), a lookup table, one or more functions (e.g., mathematical equations, software, etc.), etc. Moreover, a characteristic curve may be determined in a number of different ways depending on the implementation.

For instance, some implementations involve characteristic curves that include acoustic data (e.g., acoustic signals, harmonics information, decibel readings, etc.). The acoustic data may be collected by one or more sensors in the user interface, the conduit connected thereto and/or on or within the respiratory therapy device, such as by a microphone in acoustic communication with the airflow. The acoustic data may also be generated differently depending on the implementation. In some implementations, which are in no way intended to limit the invention, acoustic signals (such as those generated by operation of the motor, or by a speaker, etc.) may be directed along the interior of the conduit and into the user interface connected thereto. Acoustic reflections are then generated as the signals contact features of the user interface and/or conduit and detected by an acoustic sensor, such as acoustic sensor 224. These acoustic reflections may be further analyzed to determine a specific type of user interface and/or conduit that produced the detected acoustic reflections. Examples implementing analysis of acoustic signal reflections to characterize a user interface and/or conduit are described in International Publication Nos. WO 2021/250553A1 and WO2021/245637, each of which is incorporated herein in its entirety.

According to other implementations, again which are in no way intended to limit the invention, acoustic data may be collected using a microphone internal to (e.g., positioned along the interior of) the user interface, conduit connected thereto and/or the respiratory therapy device. Any vents in the user interface may be fully open (i.e., not occluded) while the air pressure is ramped across a range of values. Moreover, the different pressures may be used in combination with time, frequencies, etc., to characterize the specific type of user interface and/or conduit that are connected to a respiratory therapy device. Examples implementing methods, such as those comprising ramped pressure testing, to generate audio data for characterization of a user interface are described in the aforementioned WO 2022/219481A1.

In still other implementations, the first characteristic curve may be determined by comparing the first airflow data received to various pre-defined characteristic curves (e.g., see FIG. 8 below). For example, a system may have limited compute capabilities available, and may not be able to actually calculate a custom characteristic curve based on airflow parameter data received. The pre-defined characteristic curves may be developed over time, predetermined based on testing performed on the components of the system during manufacture, based on user input, etc. Accordingly, operation 606 may include comparing the airflow parameter data received to a variety of existing data having known relationships with specific types of user interfaces and/or conduits. Depending on the implementation, the number of pre-defined characteristic curves available to compare against the received airflow parameter data may include 2, 3, 4, 5, 10, 15, 20, 25, 30, 40, 50, 60, 70, 80, 90, 100, 1000, etc. unique characteristic curves. It should also be noted that while the present implementation has been described in the context of characteristic curves, this term is in no way intended to be limiting. For instance, rather than comparing the airflow parameter data to pre-defined characteristic curves, equivalent comparisons may be made, e.g., to a lookup table that includes data that may otherwise be represented in a characteristic curve.

In still other implementations, the first characteristic curve may be determined by actually calculating the curve. In other words, the first characteristic curve may be generated using the received first airflow parameter data, such as described in WO/2021/176426A1, which is herein incorporated by reference. For example, a system may not have access to pre-defined characteristic curves, or the airflow parameter data received may not match one of the available pre-defined characteristic curves sufficiently (e.g., less than 75% of the received airflow parameter data fits on any of the pre-defined characteristic curves). It follows that in such implementations the amount of airflow parameter data received is preferably sufficient to generate an accurate representation of how the various components operate in different settings. The calculated characteristic curve may be improved over time as additional airflow parameter data is received such that the accuracy by which the curve represents the respiratory therapy system (and the components included therein) is continually improving.

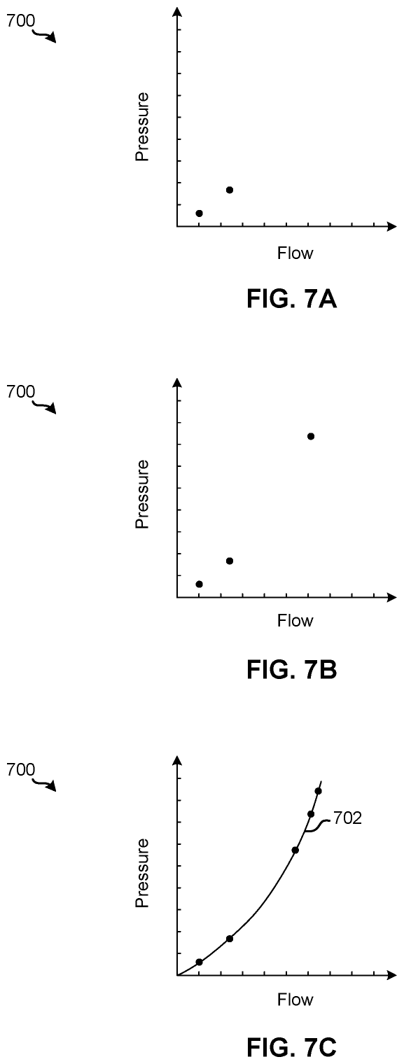

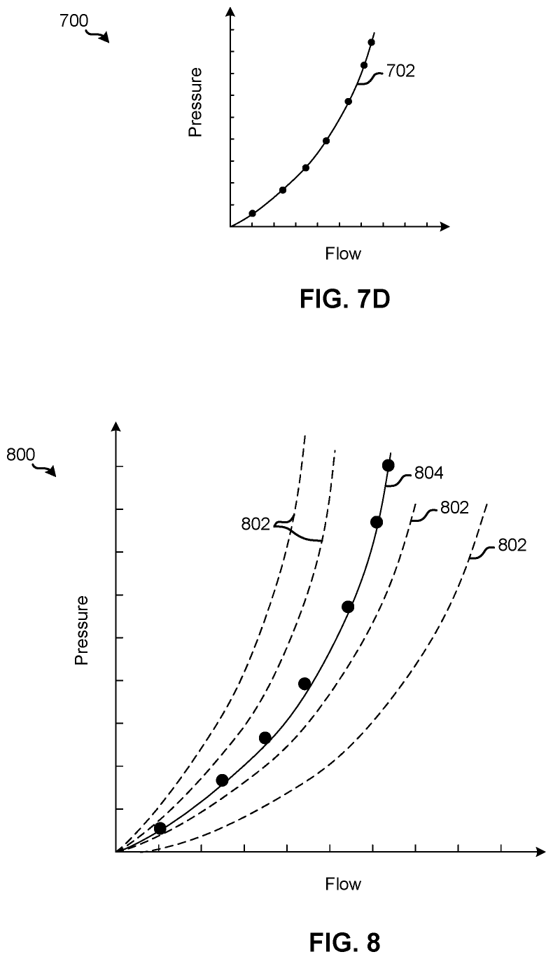

According to an example, as the speed of the motor in the respiratory therapy device is controlled to change over time, the rich data received from various sensors associated with the respiratory therapy device may be used to create data points that progressively improve the accuracy of the characteristic curve (e.g., see FIGS. 7A-7D below). It follows that in some instances, calculating the characteristic curve includes fitting a quadratic curve through at least two Cartesian points corresponding to the respective characteristic curve. While the values associated with the Cartesian points may vary, in some implementations each Cartesian point includes a pressure value and flow value. However, it should again be noted that while the present implementation has been described in the context of characteristic curves, this term is in no way intended to be limiting. For instance, rather than calculating the characteristic curves, equivalent calculations may be made, e.g., determining values for a lookup table that includes data that may otherwise be represented in a characteristic curve.

The determined first characteristic curve may be used to evaluate performance of the respiratory therapy system and the various components included therein during use. As noted above, the first characteristic curve provides a representation of how the respiratory therapy device, as presently configured, is expected to operate. The first characteristic curve may thereby be used to extrapolate the specific configuration of the respiratory therapy device. For instance, the first characteristic curve may indicate the specific user interface that is coupled to the respiratory therapy device. As noted above, while at least some air may vent intentionally from the system (e.g., see vents 372 of FIG. 3A-3B), in some situations air may inadvertently leak from a user's mouth and/or the user interface during use. This leakage may result from a poor seal between the user interface and the user's face, damage to the user interface and/or conduit, inaccurate operating conditions, etc. Thus, by determining the user interface, conduit, and/or any combination of components, pressure and flow deviations from the predetermined characteristic curve associated with user interface, conduit, and/or any combination of components, can be indicative of unintentional leak. This provides an accurate understanding of how the respiratory therapy device should perform in a variety of different scenarios, as well as the operating conditions to implement in order to achieve the desired performance.

Proceeding to operation 608, a second airflow is generated while the user interface is donned by the user. As noted above, in some implementations, a request may be sent to the user, prompting them to don the user interface. In other implementations, various data received from sensors associated with the respiratory therapy device may be used to automatically (e.g., dynamically) determine that the user interface has been donned by the user. For example, one or more sensor readings may indicate that a user has donned the user interface in preparation for a sleep session. It follows that, while the first airflow may be generated by operating the motor, humidifier, etc., in a manner that produces properties that would otherwise be undesirable to a user donning the user interface, the second airflow is preferably suitable for a user donning the user interface while awake and/or during a sleep session. The second airflow may thereby be generated by operating the motor, humidifier, etc., according to one or more nominal (e.g., predetermined) operating conditions while the user interface is donned by the user during a sleep session.

Method 600 further includes receiving second airflow parameter data associated with the second airflow—see operation 610. In some implementations, it is preferred that the second airflow parameter data received is the same as, or similar to, the type(s) of airflow parameter data received in the first airflow parameter data. This allows for the second airflow parameter data to expand upon the first airflow parameter data and improve the accuracy by which the user interface and/or conduit may be identified. As noted above, estimations can be improved upon as additional information is received. In other implementations the second airflow parameter data may include different types of airflow parameter data than the first airflow parameter data. However, it should be noted that the second airflow parameter data may be received in a same or similar manner to any of the implementations described above with respect to operation 604. Accordingly, the second airflow data may include any one or more of acoustic data, flow data, pressure data, impedance data associated with the second airflow passing through the conduit, etc.

Looking now to operation 612, there method 600 includes determining a second user interface prediction. In other words, operation 612 includes using the airflow parameter data received in operation 610 and/or information associated with the second airflow(s) generated in operation 608 to make a second (e.g., updated) prediction as to the type of user interface that is coupled to the respiratory therapy device via the conduit. This second user interface prediction may be made using any desired information, processes, inputs, etc., e.g., according to any of the implementations described above with respect to operation 606 above.

For instance, a second characteristic curve may be determined for the user interface and the conduit. As noted above, in some implementations a second characteristic curve may be used to represent how the user interface and/or conduit function while the respiratory therapy device is donned by the user and currently in use, with respect to two or more characteristics. Accordingly, while the first characteristic curve may be determined using information received while the user interface was not donned by a user, the second characteristic curve may be determined after a sleep session has started. In other words, the second characteristic curve may be determined while the user interface is donned by the user and the respiratory therapy device is operational (e.g., powered and functioning).

The second characteristic curve may be a pressure versus flow curve, an acoustic reflection curve, etc. In some implementations, it is preferred that the second characteristic curve is of a same type as the first characteristic curve. Accordingly, determining the second characteristic curve may be based, at least in part, on the first characteristic curve. For example, in implementations where the second characteristic curve is selected from a number of pre-defined curves, at least some of the pre-defined curves may be preemptively excluded from consideration based on the first characteristic curve. However, in other implementations the second characteristic curve may be a different type than the first characteristic curve.

As noted above, the second characteristic curve may be determined by comparing the first and/or second airflow data received to various pre-defined characteristic curves (e.g., see FIG. 8 below). The pre-defined characteristic curves may be developed over time, predetermined based on testing performed on the components of the system during manufacture, or based on user input, etc. Accordingly, operation 612 may include comparing the received airflow parameter data to a variety of existing data having known relationships with specific types of user interfaces and/or conduits. Depending on the implementation, the number of pre-defined characteristic curves available to compare against the received first and/or second airflow parameter data may include 2, 3, 4, 5, 10, 15, 20, 25, 30, 40, 50, 60, 70, 80, 90, 100, 1000, etc. unique characteristic curves. It should also be noted that while the present implementation has been described in the context of characteristic curves, this term is in no way intended to be limiting. For instance, rather than comparing the airflow parameter data to pre-defined characteristic curves, equivalent comparisons may be made, e.g., to a lookup table that includes data that may otherwise be represented in a characteristic curve.

In still other implementations, the second characteristic curve may be determined by actually calculating the curve. In other words, the second characteristic curve may be generated using the received second airflow parameter data, similar to described above in respect of the first airflow parameter data. It follows that in such implementations the amount of airflow parameter data received is preferably sufficient to generate an accurate representation of how the various components operate in different settings. The calculated characteristic curve may be improved over time as additional airflow parameter data is received such that the accuracy by which the curve represents the respiratory therapy device (and the components included therein) is continually improving. In some implementations the first characteristic curve may thereby serve as a template that is improved upon to develop the second characteristic curve. In other words, the process of determining the second characteristic curve for the user interface and the conduit is based, at least in part, on the first characteristic curve.

From operation 612, method 600 advances to operation 614 which includes characterizing the user interface. This characterization is preferably based on the first and/or second user interface predictions, and involves identifying information associated with the user interface, conduit, cushion, etc. As noted above, characterization curves represent how a specific configuration of a respiratory therapy device performs. In other words, the specific type of user interface, conduit, cushion size, etc., that produced the data points in a given characteristic curve are retained and correlated with the characteristic curve. Thus, once a characteristic curve has been determined (e.g., calculated, selected from a plurality of pre-defined characteristic curves, etc.) for an implementation, the characteristic curve(s) may be able to provide an insight as to the specific components that are in use. By monitoring performance and creating correlations between data points, method 600 is thereby able to automatically deduce the type of user interface, conduit, cushion size, etc. being used by a user, without any direct input from the user (e.g., other than potentially using the respiratory therapy device).

This automatic detection further allows for implementations to dynamically adjust performance of the system in response to detecting changes to the respiratory system components being used. For example, a direct user interface may be removed from the respiratory therapy device by a user, and replaced with an indirect user interface, e.g., based on personal preference of the user, changing sleeping conditions, a time of day, amount of use, etc. The newly introduced indirect user interface will return airflow parameter data that differs (at least somewhat) from the airflow parameter data used to determine the first, second, third, etc., characteristic curves. These differences may be detected by identifying inconsistencies between expected and actual airflow parameter data, or previous and current airflow parameter data. It follows that airflow parameter data received during use of the respiratory therapy device may constantly, periodically, occasionally, etc., compared to the expected data based on the characteristic curves. While not specifically depicted in FIG. 6A, method 600 may thereby monitor performance in the background and automatically identify changes to the configuration of the respiratory therapy device. This allows for performance of the respiratory therapy device to be adjusted in real time such that the quality of therapy received and/or sleep experienced by the user during a sleep session is optimized.

As mentioned above, the characteristic curves and/or characterization of the components in the respiratory therapy device may further be used to evaluate performance of the respiratory therapy device during use in some implementations. FIG. 6B illustrates an exemplary sub-method 620 of operations for determining unintentional leak from a user interface, which may be performed based on at least some of the information received and/or determined in method 600. In some implementations the operations in sub-method 620 may be performed in the background by a same or different element or aspect of the system 100 (FIGS. 1-2B) as used to perform one or more steps of method 600. However, any one or more of the operations in sub-method 620 may be performed by any desired type of component (e.g., processor).

Looking to FIG. 6B, operation 622 includes determining a third characteristic curve for the user interface and the conduit. As mentioned above, a characteristic curve may be determined in a number of different ways depending on the implementation. For instance, in some situations it may be advantageous to select a characteristic curve from a plurality of pre-defined characteristic curves. For example, a system may have limited compute capabilities available, and may not be able to actually calculate a custom characteristic curve based on airflow parameter data received. Though in other situations, it may be desirable to calculate a new characteristic curve using known information, e.g., such as received airflow parameter data. For example, a system may not have access to pre-defined characteristic curves, or the airflow parameter data received may not match any of the available pre-defined characteristic curves sufficiently (e.g., less than 90% of the received airflow parameter data fits on any of the pre-defined characteristic curves). In still other implementations, the third characteristic curve may be a predetermined (e.g., template) characteristic curve for a particular user interface or a particular user interface and conduit combination. For instance, the third characteristic curve may be a preferred characteristic curve for the characterized user interface.

The type of characteristic curve determined for the third characteristic curve may also vary depending on the implementation. For instance, in some implementations it may be advantageous that the third characteristic curve is a same type of curve as the first and/or second characteristic curves. Again, this may allow for the characteristic curves to progressively improve the accuracy by which the performance of the system is understood. However, the third characteristic curve may be a different type of curve than the first and/or second characteristic curves. For example, using different types of characteristic curves may provide a more well-rounded understanding of the system and the various components included therein. It follows that the third characteristic curve may be a pressure versus flow curve, an acoustic reflection curve, etc., or any other desired type of curve that represents data corresponding to performance of at least the respiratory therapy device.

By incorporating the first and/or second characteristic curves, in addition to airflow parameter data received during operation of the respiratory therapy device, the third characteristic curve may serve as a best representation of performance. Operation 624 further includes determining unintentional leak from the user interface. Unintentional air leakage from the user interface may result from incorrectly donning the user interface, operating settings that are incorrect for a given implementation, damage to the components of the respiratory therapy device, etc. The third characteristic curve may thereby be used, at least in part, to determine the presence of any such unintentional leak. This determination of whether unintentional leakage is present may be performed during an ongoing sleep session (e.g., a same sleep session that resulted in the second airflow parameter data above), a next sleep session, future sleep sessions, future general use of the respiratory therapy system by the user, etc.

Unintentional leakage may be identified by comparing airflow parameter data received while operating the respiratory therapy device, with data included in the third characteristic curve. Any identified differences between the expected performance illustrated in the third characteristic curve and the received airflow parameter data may be attributed to or identified as unintentional leakage. Although not illustrated in FIG. 6B, determined unintentional leak may be output to a user in a notification or warning message (e.g., displayed on a user interface, delivered to a mobile device of the user over a network, displayed or delivered to a third party such as a physician or home medical equipment provider, etc.), sent to a manufacturer as training data, saved in local memory for additional processing, used to dynamically update operating conditions of the respiratory therapy device, etc. For example, the first and second characteristic curves, and/or the characterization of the user interface, may be used to determine whether the operating parameter of the respiratory therapy device, such as the speed of the motor and/or the settings of the humidifier, should be adjusted to improve the quality of sleep experienced by the user donning the user interface.

In some implementations, after determining, and optionally quantifying an amount of, unintentional leakage, the speed of the motor of the respiratory therapy device can be adjusted (e.g., increased) by discrete increments to compensate for the unintentional leakage. For example, the user can set a pressure level that should be maintained in the conduit 140 and/or the user interface 120 (i.e., the pressure at which air is delivered to the patient based on recommended therapy). Unintentional leakage from the air circuit and/or user interface, or indeed from the mouth of the user (such as mouth leak that may be experienced by users of nasal/pillows masks) can provide a discrepancy between the realized pressure within the user's prescribed pressure level and the actual pressure in the conduit 140 and/or the user interface 120. The speed of the motor can be adjusted in set increments such that the pressure within the air circuit and/or the user interface matches the prescribed therapeutic pressure.

In some implementations, the pressure sensor 212 can be used to measure, or estimate, real-time pressure within the air circuit and/or the user interface to determine when to stop adjustments to the speed of the motor.

In some implementations, the flow rate sensor 214 can be used to provide real-time air flow rate information to determine when to stop adjusting the speed of the motor once a corresponding increase in air flow rate compensates for the determined unintentional leakage. In some implementations, the humidifier 160 can be used to adjust humidity levels (of the flow of air provided to the user) in response to adjustments to the flow rate, pressure levels and/or speed of the motor. One or more of the above-described adjustments to operating settings of the respiratory therapy system can be determined/implemented in order to improve the quality of sleep experienced by the user and/or quality (e.g., effectiveness) of therapy provided to the user.

As mentioned above, generating airflows while the user interface is not donned by the user allows for the respiratory therapy device to be operated without concern for a comfort level of a user. A wide range of operating conditions may thereby be implemented such that a valuable amount of airflow parameter data is received. Components such as a motor in the respiratory therapy device may thereby be set to operate at one or more speeds that produce noise, heat, vibrations, air properties, etc., that would otherwise be undesirable to a user donning the user interface. This allows for the system to collect information associated with a variety of different operating settings that can be used to characterize the various components included in the system, e.g., as will soon become apparent.

Looking now to FIG. 6C, a method 630 for operating a motor of a respiratory therapy device at a variety of different speeds is illustrated. While the configuration of the respiratory therapy device may differ depending on the implementation, method 630 has been presented below in the context of a system having a user interface that is coupled to a respiratory therapy device, optionally via a conduit (e.g., see FIGS. 2A-2B). It follows that one or more steps of the method 630 can be implemented using any element or aspect of the system 100 (FIGS. 1-2B) described herein.

It should also be noted that method 630 may preferably be performed while the user interface is not donned by a user. As noted above, in some implementations, by ramping the operating speed of the motor up and/or down to a number of speeds allows for an improved understanding of the components that are included in, or coupled to, a respiratory therapy device and how they operate. By generating the first airflow while the user interface is not donned by the user, the respiratory therapy device may be operated without concern for a comfort level of the user. This allows for the system to collect information associated with a variety of different operating settings that can be used to better characterize the various components included in the system.

Looking now to operation 632, an airflow is generated by causing the motor to operate at a first discrete speed. The first discrete speed may be predetermined, e.g., by a user or manufacturer, etc., a lowest speed supported by the motor, a randomly selected speed, an average of past motor speeds during use (e.g., during sleep sessions), etc. Moreover, causing the motor to operate at the first discrete speed may include sending one or more instructions to the motor itself, a controller coupled to the motor, etc.

Accordingly, airflow parameter data is received in operation 634. The airflow parameter data that is received may be collected by various sensors that are associated with (e.g., coupled to, positioned near, etc.) the respiratory therapy device, the user interface, and/or the conduit.

Operation 636 further includes using the received airflow parameter data to determine a user interface prediction. As noted above, received airflow parameter data may be used to determine a user interface prediction by identifying a characteristic curve in a number of different ways depending on the implementation. For instance, in some implementations the characteristic curve may be a pressure versus flow curve that is calculated (e.g., plotted) using the received airflow parameter data. In other instances, the characteristic curve may be an acoustic reflection curve that is selected from a plurality of pre-defined acoustic reflection curves.

It follows that the airflow parameter data received in operation 636 preferably includes data resulting from the airflow(s) generated in operation 632. In other words, the motor preferably maintains the discrete speed long enough for rich data samples associated with the given operating condition may be collected by various sensors in the respiratory therapy system. According to an example, which is in no way intended to limit the invention, a motor in the respiratory therapy device may produce a specific number of revolutions per minute for a period of time sufficient for sensors in and/or around the respiratory therapy device to collect rich data associated with how the various other components in the respiratory therapy device operate at a specific air pressure, e.g., as would be appreciated by one skilled in the art after reading the present description.

From operation 636, method 630 proceeds to operation 638. There, operation 638 includes generating a unique airflow by causing the motor to operate at a different discrete speed. In other words, operation 638 includes causing the motor to adjust its operating speed such that an airflow having different properties (e.g., pressure, humidity, flow, etc.) is produced. The updated speed at which the motor is instructed to operate may be based on the previous motor speed in some instances. For example, the updated speed may be a next discrete speed in a predetermined list of motor speeds. In other instances, the updated motor speed may be based on airflow parameter data received, user input, predetermined ranges, preferred operating settings of the respiratory therapy device, a type of user interface believed to be implemented, etc.

Operation 640 thereby includes receiving unique airflow parameter data that corresponds to the different discrete speed of the motor. The unique airflow parameter data may include any desired type of information, e.g., such as one or more of acoustic data, flow data, pressure data, and impedance data associated with the first airflow passing through the conduit, etc.

The unique airflow parameter data is also preferably rich data that can be used for further processing of the characteristic curve. As noted above, each unique operating speed of the motor is preferably maintained long enough for rich data samples associated with the given motor speed to be collected by various sensors. According to an example, which is in no way intended to limit the invention, a motor in the respiratory therapy device may produce a specific number of revolutions per minute for a period of time sufficient for sensors in and/or around the respiratory therapy device to collect rich data associated with how the various other components in the respiratory therapy device operate at a specific air pressure, e.g., as would be appreciated by one skilled in the art after reading the present description.

Operation 642 further includes using the received unique airflow parameter data to update the user interface prediction. In other words, the unique airflow parameter data received in response to causing the motor to operate at a different discrete speed, is used to improve the accuracy by which the user interface prediction represents the specific configuration of the respiratory therapy device. It follows that, as the speed of the motor in the respiratory therapy device changes over time, the rich data received from various sensors associated with the respiratory therapy device may be used to create data points that progressively improve the accuracy of the characteristic curve (e.g., see FIGS. 7A-7D below). It should be noted that the term “curve” as used herein is in no way intended to be limited by the number of data points that correspond thereto. In other words, a curve may correspond to (e.g., be defined by) a pair of data points (such as a flow value and corresponding pressure value associated with the airflow generated by the respiratory therapy device) which suffice to generate the “curve”. In some implementations, a single data point, or a pair of data points, may suffice in creating a “curve” that may be compared to a template (e.g., predetermined) curve associated with a user interface or user interface-conduit combination. Other techniques may also be used in combination with the generation of curves as described herein. For example, techniques such as a machine learning approach may use one or more data points to identify or otherwise characterize a user interface, corresponding to a user interface prediction as described herein. In such an approach, airflow parameter data may be inputted to a machine learning model, such as a machine learning model trained with user interface information and corresponding airflow parameter data, to output a user interface prediction.

Accordingly, the flowchart of method 630 returns to operation 638 from operation 642 such that the motor is adjusted to operate at yet another discrete speed. This allows for a new airflow to be produced that ultimately results in additional unique airflow parameter data to be received, which can be used to further improve the characteristic curve. It follows that operation 638, 640, 642 may be repeated in an iterative fashion any desired number of times, for a predetermined amount of time, etc. The number of times one or more of operations 638, 640, 642 are repeated may depend on a type of motor being used, user preferences, the type of characteristic curve, preferred tolerance (e.g., accuracy) levels, past performance, etc. However, it should again be noted that the motor preferably operates at each of the discrete speeds for a long enough period of time that rich airflow parameter data may be received at each of the discrete motor speeds, e.g., as would be appreciated by one skilled in the art after reading the present description.

The speed at which the motor operates during method 630 may also be ramped up and/or down to any number of desired speeds. As noted above, this allows for an improved understanding of the components that are included in, or coupled to, a respiratory therapy device and how they operate.

As noted above, in some implementations a characteristic curve is determined by actually calculating the curve. In other words, a characteristic curve may actually be generated using received airflow parameter data. It follows that in such implementations the amount of airflow parameter data received is preferably sufficient to generate an accurate representation of how the various components operate in different settings. The calculated characteristic curve may be improved over time as additional airflow parameter data is received such that the accuracy by which the curve represents the respiratory therapy device (and the components included therein) is continually improving.

For instance, looking now to FIGS. 7A-7D, a pressure versus flow graph 700 is depicted at various stages of calculating a characteristic curve 702 according to an illustrative implementation, which is in no way intended to limit the invention. Accordingly, any one or more of the progressions of graph 700 illustrated in FIGS. 7A-7D may be used to calculate at least a portion of a characteristic curve in any of the implementations herein.

FIG. 7A shows graph 700 as including only 2 data points plotted thereon. As noted above, the graph 700 is a pressure versus flow graph, and therefore each of the data points included in FIG. 7A may be a Cartesian point having a pressure and an airflow value. Data corresponding to the 2 data points plotted in FIG. 7A may be received in response to the respiratory therapy device being operated in 2 or more different settings (e.g., motor operating at different speeds). Another data point plotted on graph 700 in FIG. 7B may result from airflow parameter data received in response to the respiratory therapy device being operated in yet another setting.

Airflow parameter data may be received and plotted on the graph 700 and from which a characteristic curve 702 may be determined (e.g., calculated) and applied. The characteristic curve may be determined using any processes that would be apparent to one skilled in the art after reading the present description. For example, the plotted airflow parameter data and one or more quadratic questions may be used to determine a characteristic curve that intersects each of the plotted points. This characteristic curve may be represented and/or expressed mathematically, graphically, logically, verbally, etc., or in any other manner. In other words, a general way of representing the various airflow parameter data is preferably determined.

While the characteristic curve 702 is shown plotted on the graph 700 of FIG. 7C, improvements may be made thereto as additional airflow parameter data is received. Accordingly, the graph 700 seen in FIG. 7D includes additional plotted points that increase the accuracy by which the characteristic curve 702 may be represented.

As noted above, calculating a characteristic curve may not be a preferred (e.g., viable) option in some implementations. For example, compute power may be limited and/or the type of airflow parameter data received may be of a specific type (e.g., such as acoustic data for an acoustic reflection curve). Thus, rather than calculating the characteristic curve, it may be selected from a plurality of pre-defined curves based on limited airflow parameter data, e.g., one or more data points (e.g., a pressure and flow data point).

Looking to FIG. 8, a graph 800 having several pre-defined curves 802 is depicted according to an illustrative implementation, which is in no way intended to limit the invention. Accordingly, any one or more of the pre-defined curves 802 illustrated in FIG. 8 may be selected as representing the airflow parameter data received.

Although the pre-defined curve 804 that is ultimately selected may not be a perfect representation of the airflow parameter data received, it is preferably a closest match. For example, selected curve 804 does not perfectly intersect all of the data points plotted on the graph 800. The process of selecting one of the pre-defined curves may thereby involve determining how closely a curve matches the available data points. For example, a classification-based machine learning algorithm may use a KNN (K-Nearest Neighbor) technique by classifying based on a Euclidean distance of the airflow parameter value from the parameter data defining the pre-defined curves. In some implementations, a characteristic curve may be calculated for the airflow parameter data received, and compared to available pre-defined curves. Each of the pre-defined curves may correspond to a known configuration of the respiratory therapy system, and therefore may be able to provide valuable information in characterizing the type of user interface, conduit, cushion, etc., being used.

It should be noted again that any of the characteristic curves included herein may the same, similar, or different types compared to each other. The characteristic curves included herein may also be created in a number of different ways depending on the particular implementation. For instance, any one of the first, second, third, etc., characteristic curves may be selected from the plurality of pre-defined characteristic curves, calculated in real-time based on received airflow parameter data, etc. Moreover, any of the characteristic curves may be used in the process of determining subsequent characteristic curves. For example, determining the second characteristic curve may be based at least in part on the first characteristic curve, determining the third characteristic curve may be based at least in part on the first and/or second characteristic curves, etc.

The numbering used herein, e.g., such as the numbering of the characteristic curves, is also in no way intended to be limiting. Rather, any desired type of naming convention may be used to distinguish between the various components, characteristic curves, received airflow parameter data, airflows, etc. Moreover, the automatic detections achieved in the various implementations herein may be applied to other systems and/or components. For example, one or more of the processes described herein may be used to automatically detect a current power supply of the respiratory therapy device and dynamically adjust operating conditions in real time based on the amount and quality of power available for use. In some implementations, the various components included in a respiratory therapy device and/or coupled thereto may include an electrically conductive connection configured to uniquely identify the respective components. Thus, as components such as a user interface is disconnected from the conduit, the respiratory therapy system may be able to detect that the user interface has been disconnected. The system may also be able to detect a different user interface that is coupled to the conduit thereafter in response to the unique electrical characteristics (e.g., resistance, supply voltage, conductance, etc.) associated with the electrically conductive connection of the newly coupled user interface.

One or more elements or aspects or steps, or any portion(s) thereof, from one or more of any of claims 1 to 60 below can be combined with one or more elements or aspects or steps, or any portion(s) thereof, from one or more of any of the other claims 1 to 60 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

What is claimed is:

1. A method for characterizing a user interface, the user interface being coupled, via a conduit, to a respiratory therapy device in a respiratory therapy system, the method comprising:
generating, while the user interface is not donned by a user, a first airflow from the respiratory therapy device to the user interface by operating a motor of the respiratory therapy device;
receiving first airflow parameter data associated with the first airflow;
determining, based at least in part on the first airflow parameter data, a first user interface prediction;
generating, while the user interface is donned by the user, a second airflow from the respiratory therapy device to the user interface by operating the motor of the respiratory therapy device;
receiving second airflow parameter data associated with the second airflow;
determining, based at least in part on the second airflow parameter data, a second user interface prediction; and
characterizing the user interface based at least in part on the first user interface prediction and the second user interface prediction.

2. The method of claim 1, wherein the determining the second user interface prediction is also based at least in part on the first user interface prediction.

3. The method of claim 1, further comprising determining, based at least in part on the characterized user interface, a third user interface prediction.

4. The method of claim 3, wherein the third user interface prediction is a third characteristic curve selected from a plurality of pre-defined characteristic curves.

5. The method of claim 1, wherein the first user interface prediction is a first characteristic curve selected from a plurality of pre-defined characteristic curves.

6. The method of claim 1, wherein the second airflow is generated while the user interface is donned by the user during a sleep session, wherein the determining the second user interface prediction includes calculating a second characteristic curve after the sleep session has started.

7. The method of claim 6, wherein calculating the second characteristic curve includes fitting a quadratic curve through at least two Cartesian points corresponding to the respective characteristic curve, wherein each of the at least two Cartesian points has a pressure value, a flow value, or impedance value.

8. The method of claim 6, further comprising using the first characteristic curve to determine unintentional leak from the user interface during at least a first portion of the sleep session, wherein the determining the second characteristic curve is also based at least in part on the first characteristic curve.

9. The method of claim 1, wherein the first airflow parameter data and the second airflow parameter data each include one or more airflow parameters selected from the group consisting of: acoustic data, flow data, pressure data, and impedance data associated with the first airflow passing through the conduit.

10. The method of claim 1, wherein the characterizing of the user interface includes characterizing a user interface and conduit combination.

11. The method of claim 1, wherein the generating, while the user interface is not donned by the user, the first airflow includes operating the motor at a first speed, wherein the generating, while the user interface is donned by the user, the second airflow includes operating the motor at a second speed, the second speed being greater than the first speed.

12. The method of claim 11, wherein the first speed is between about 3,000 revolutions per minute (RPM) and about 12,000 RPM.

13. The method of claim 1, wherein the generating, while the user interface is not donned by the user, the first airflow includes operating the motor at a plurality of discrete speeds, wherein operating the motor at one or more of the plurality of discrete speeds causes operating conditions that would be undesirable to the user while donning the user interface.

14. A respiratory therapy system comprising:
a respiratory therapy device;
a conduit;
a user interface, the user interface being coupled, via the conduit, to the respiratory therapy device;
a memory device having stored thereon machine-readable instructions; and
a control system including one or more processors configured to execute the machine-readable instructions to:
generate, while the user interface is not donned by a user, a first airflow from the respiratory therapy device to the user interface by operating a motor of the respiratory therapy device;
receive first airflow parameter data associated with the first airflow;
determine, based at least in part on the first airflow parameter data, a first user interface prediction;
generate, while the user interface is donned by the user, a second airflow from the respiratory therapy device to the user interface by operating the motor of the respiratory therapy device;
receive second airflow parameter data associated with the second airflow;
determine, based at least in part on the second airflow parameter data, a second user interface prediction; and
characterize the user interface based at least in part on the first user interface prediction and the second user interface prediction.

15. The respiratory therapy system of claim 14, wherein the determining the second user interface prediction is also based at least in part on the first user interface prediction.

16. The respiratory therapy system of claim 14, wherein the one or more processors are further configured to execute the machine-readable instructions to determine, based at least in part on the characterized user interface, a third user interface prediction.

17. The respiratory therapy system of claim 16, wherein the first user interface prediction is a first characteristic curve selected from a plurality of pre-defined characteristic curves and wherein the third user interface prediction is a third characteristic curve selected from the plurality of pre-defined characteristic curves.

18. The respiratory therapy system of claim 14, wherein the first airflow parameter data and the second airflow parameter data each include one or more airflow parameters selected from the group consisting of: acoustic data, flow data, pressure data, and impedance data associated with the first airflow passing through the conduit.

19. The respiratory therapy system of claim 14, wherein the control system including the one or more processors is further configured to execute the machine-readable instructions to determine an adjustment to operating settings of the respiratory therapy system based on the characterization of the user interface, wherein the adjustment is configured to improve a quality of therapy provided to the user and/or sleep experienced by the user during the sleep session.

20. The respiratory therapy system of claim 19, wherein the adjustment relates to one or more of (i) flow rate, (ii) pressure, and (iii) humidification levels of the airflow generated by the respiratory therapy device.
